(19) United States
(12) Patent Application Publication
(10) Pub. No.: US 2005/0002975 A1
(43) Pub. Date: Jan. 6, 2005

(54) Structures and methods for delivering topical compositions

(76) Inventor: Densen Cao, Sandy, UT (US)

Correspondence Address: Parsons Behle & Latimer;Suite 1800, 201 South Main Street, P.O. Box 45898, Salt Lake City, UT, 84111, US

(21) Appl. No.: 10/862,052

(22) Filed: Jun. 4, 2004

Related U.S. Application Data
(60) Provisional application No. 60/476,773, filed on Jun. 6, 2003.

(51) Int. Cl. A61K 9/14

(52) U.S. Cl. 424/401; 424/489

(57) ABSTRACT

A system for topically administering a chemical agent to a human body. The chemical agent may be mixed with a carrier and the form a tangible application device. The carrier may be a compound or a physical structure such as an absorbent material.

### CROSS REFERENCE TO RELATED APPLICATIONS

Priority and benefit under 35 U.S.C. § 120 to U.S. Provisional Patent Application Ser. No. 60/476,773 is hereby claimed.

### BRIEF DESCRIPTION OF DRAWINGS

FIG. 1 is a perspective view of a chemically impregnated patch or strip suitable for topical application to the human body.

FIG. 2 is a perspective view of a strip in illustrating example desired thickness of the material.

FIG. 3 is an illustration of the application of a completed strip applied to the tooth structure.

### DETAILED DESCRIPTION

Topical compositions may be delivered to a human patient by use of a carrier or substrate that is impregnated with a chemical agent such as a medicine, pain killer, tooth whitener, etc. The carrier impregnated with a chemical agent may be placed on a desired surface on a patient, such as on the teeth, on the gums, on the skin, etc. so that the chemical agent will contact the surface on the patient in order to impart a desired effect thereto.

Structures and methods which carry out these functions are disclosed. They are to be distinguished from using a substrate and a layer of a chemical agent as distinct units. Impregnation of the chemical agent into the carrier is a materially different delivery mechanism than use of a substrate and a layer of chemical agent on top of it.

The technology of this disclosure allows chemical agents to be applied in a topical manner such as by homogeneously or otherwise incorporating or embedding said chemical agents into the application device, or carrier. Said chemical agents may be incorporated or embedded into various application devices or carriers, such as an applications device having a composition containing one or more desired polymer materials in concentrations of 0.01% to 99.9% by weight. Depending on the polymer used, the molecular weight of the polymer material can have a weight average molecular weight of any appropriate amount for the application, such as at least 100, at least 1,000, at least 10,000, at least 100,000 or at least 1,000,000 as determined according to the gel permeation chromatography method.

Suitable materials for the carrier include polyvinyl alcohol (PVA) and its partially hydrolyzed polyvinyl acetate equivalents, polyvinyl acetate (PVAc), polyvinylpyrrilidone (PVP), polyethylene glycol (also referred to as poly oxyethylene), polypropylene glycol, polyacrylic acid and its derivatives, carboxymethyl cellulose, related cellulosic compounds, xantham gum, related resin gums, gels, including gels formed by an A-B copolymer or an A-B-A triblock copolymer plasticized with an appropriate plasticizing agent so that the gel is generally non-flowable at room temperature. Other carriers may be employed with a composition of silicas, silicates, and silanes. Another alternative is to embed the chemical agents into a paper, cloth or other absorbent material as a carrier that will later release the chemical agents when desired.

An example structure and method of incorporation or embedding of a chemical agent into a carrier patch or strip is to prepare a polymer composition in a liquid state, to which is added the chemical agent of choice. The polymer composition incorporating the chemical agent is then converted to a solid state to form the application device. The application device is then applied topically to a number of structures of the human body for the purpose of releasing the chemical agent onto or into the structure of the body. This represents a substantially new technology over current technologies in that the chemical agent to be delivered is not applied as a separate composition to the polymer delivery device, but is homogeneously dispersed or embedded within the polymer delivery device, creating a delivery system that is physically one composition. Said polymer device (carrier) may be used independently, or combined with an additional laminate backing. Said polymer device may be used independently or in conjunction with other topical application systems or as a dual or multi-component system.

Another example does not rely on chemical incorporation of the chemical agent into the carrier, but uses a carrier of absorbent material such as paper or cloth. The absorbency of the carrier will tend to pick up and hold the chemical agent for later leaching out into a desired surface of the human body to carry out a treatment. The carrier may be super-saturated with the chemical agent such that the chemical agent readily flows or leaches out of it, or it may be only fully or partially saturated in order to provide a slow release of the chemical agent to a human subject over time.

One example carrier uses polyvinyl alcohol (PVA) as at least one of the polymer materials for the delivery device. Two or more polymers may be mixed or associated to form a carrier. Another example uses an absorbent or hyrophillic paper to absorb, hold and later release the chemical agent. Another example uses an absorbent or hydrophillic cloth to absorb, hold and later release the chemical agent.

The carrier may be used to store and later release many desired chemical agents for many different purposes. One example is the delivery and the use of peroxides such as hydrogen peroxide, carbamide peroxide, and/or sodium peroxide as the chemical agent, to be delivered to tooth structures for the purpose of whitening teeth. Another example is delivery of lidocaine, benzocaine, and/or other anesthetic pharmaceuticals as the chemical agent, for the purpose of delivering said anesthetics to injured or irritated structures of the body for the purpose of reducing pain in said affected structures. Another example uses bacitracin and other antibacterial and/or antimicrobial pharmaceuticals as the chemical agent, for the purpose of delivering said other antibacterial and/or antimicrobial pharmaceuticals to injured or irritated structures of the body for the purpose of preventing or eliminating infection by foreign materials in said affected structures.

### EXAMPLE 1

A delivery device for the purpose of whitening teeth is prepared. A polymer delivery material of the following composition can be prepared:

| Component | % by weight |
|---|---|
| Water | 0.01-95.00 |
| PVA | 0.01-95.00 |
| Glycerin | 0.01-50.00 |

These components are processed to form a homogenous liquid composition, to which can be added hydrogen peroxide in concentrations of 0.01% to 40.00% by weight of the total composition. The combined composition is mixed to create a homogeneous mixture. The mixture is then deposited onto a large flat surface, which can incorporate a release backing. The mixture is spread out across the flat surface to form a thin layer with a minimum thickness of 1 micrometer. The mixture is then allowed to remain exposed to air until the mixture changes to a solid form. At this point the solid film that is formed may be released from the flat surface, or subdivided into single-application segments.

A composition as outlined was prepared using water of a concentration of 56% by weight, PVA of a concentration of 12% by weight, glycerin of a concentration of 10% by weight, and a 50% by weight solution of hydrogen peroxide in water of a concentration of 22% by weight. The mixture was then processed as outlined above to create the desired device.

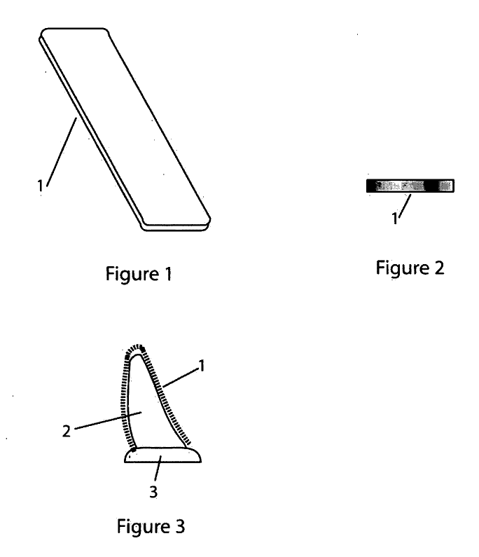

With the above technical disclose in mind, reference is now made to the figures. FIG. 1 is a perspective view of an example patch or strip in accordance with principles herein in a possible geometric shape 1 as would be suitable for topical application to the human body. FIG. 2 is perspective view of an example strip in accordance with the principles herein 1 illustrating example desired thickness of the material, said thickness indicated above as an example minimum of 1 micrometer. FIG. 3 is an illustration of the application of an example completed strip 1 as prepared according to Example 1 and applied to the tooth structure 2. The geometric shape of 1 can be adjusted such that 1 does not come in contact with gum tissue 3 at the base of tooth structure 2. It is envisioned that the geometric shape of the strip 1 could be such that an individual tooth 2 or an array of of several teeth can be treated simultaneously by the application of the strip 1. The reader should understand that a patch or strip impregnated with a desired chemical composition may be of any size or shape, as desired, and may include one or more chemical composition impregnated within it. The chemical composition may be any chemical composition having a potentially beneficial effect on a human, such as a topical anesthetic, a topical antibiotic, a topical lubricant, a lotion, vitamins, nutraceuticals, anti-fungals, a dental bleach, or any other desired chemical composition.

The devices and methods may be embodied in other specific forms without departing from their spirit or essential characteristics. The described embodiments are to be considered in all respects as only illustrative, and not restrictive. The scope of the invention is, therefore, indicated by the appended claims, rather than by the foregoing description. All changes which come within the meaning and range of equivalency of the claims are to be embraced within their scope.

1. A topical composition delivery system comprising:
a carrier,
a chemical agent,
said chemical agent being impregnated into said carrier,
said agent-impregnated carrier having a tangible physical form that may be handled and placed in a desired location on the human body in order to permit said chemical agent to move from said carrier to the human body for treatment thereof, and
said carrier being capable of releasing said chemical agent when in contact with a human body.

2. A system as recited in claim 1 wherein said chemical agent is selected from the group consisting of medicine, pain killers, tooth whiteners, lotions, anesthetic, antibiotics, vitamins, nutraceuticals, and anti-fungals.

3. A system as recited in claim 1 wherein said chemical agent is homogeneously incorporated into said carrier.

4. A system as recited in claim 1 wherein said carrier includes a polymer.

5. A system as recited in claim 4 wherein said polymer has a molecular weight of lat least 1,000,000 when measured according to the gel permeation chromatography method.

6. A system as recited in claim 4 wherein said polymer has a molecular weight of lat least 100,000 when measured according to the gel permeation chromatography method.

7. A system as recited in claim 4 wherein said polymer has a molecular weight of lat least 1,000 when measured according to the gel permeation chromatography method.

8. A system as recited in claim 1 wherein said carrier includes a material selected from the group consisting of polyvinyl alcohol (PVA), partially hydrolyzed polyvinyl acetate equivalents, polyvinyl acetate (PVAc), polyvinylpyrrilidone (PVP), polyethylene glycol, poly oxyethylene, polypropylene glycol, polyacrylic acid and its derivatives, carboxymethyl cellulose and related cellulosic compounds, xantham gum, related resin gums, gels, including gels formed by an A-B copolymer or an A-B-A triblock copolymer plasticized with an appropriate plasticizing agent so that the gel is generally non-flowable at room temperature, silica, a silicate, silanes, paper, cloth and absorbent material.

9. A system as recited in claim 1 wherein said carrier includes material selected from the group consisting of fiber, paper, cloth and absorbent materials which hold said chemical agent for later application to a human body.

10. A system as recited in claim 1 wherein said carrier includes PVA and an absorbent material.

11. A system as recited in claim 1 wherein said chemical agent is a dental bleach.

12. A system as recited in claim 11 wherein said chemical agent includes a peroxide.

13. A system as recited in claim 12 wherein said peroxide is present in said chemical agent in an amount of up to about 40% by weight.

14. A method for making a system for applying a topical composition to a human being comprising the following steps:
preparing a polymer composition in a liquid state,
adding a selected chemical agent to said polymer composition, and
convert said polymer with said chemical agent to a solid state to form an application device.

15. A method as recited in claim 14 further comprising the step of applying said application device to a human body for the purpose of releasing said chemical agent into a human body.

16. A method as recited in claim 14 wherein said chemical agent is homogeneously dispersed in said application device.
